(19) United States
(12) Patent Application Publication
(10) Pub. No.: US 2019/0000794 A1
(43) Pub. Date: Jan. 3, 2019

(54) TRANSMUCOSAL CANNABINOID FORMULATION INCLUDING A CHITOSAN EXCIPEINT

(71) Applicant: Daniel S. Tanaka, Hopewell Junction, NY (US)

(72) Inventor: Daniel S. Tanaka, Hopewell Junction, NY (US)

(21) Appl. No.: 15/855,072

(22) Filed: Dec. 27, 2017

(51) Int. Cl. A61K 31/352; A61K 47/36; A61K 47/12; A61K 47/44; A61K 9/00

(52) U.S. Cl. CPC A61K 31/352; A61K 47/36; A61K 9/006; A61K 47/44; A61K 47/12

(57) ABSTRACT

A cannabinoid formulation for transmucosal delivery includes a first excipient comprising shea butter containing at least 40% oleic acid, at least 20% steraic acid, at least 3% linoleic acid, and at least 2% palmitic acid, a second excipient comprising soy lecithin and a third excipient that is a glycolipid precursor. In one embodiment, the glycolipids precursor is a bioadhesive polysaccharide composed of randomly distributed β-(1→4)-linked D-glucosamine and N-acetyl-D-glucosamine to improve mucosal and cellular permeability. Preferably the polysaccharide is derived from naturally occurring chitosan. In one embodiment, the polysaccharide is naturally occurring chitosan having a concentration of between 1-20 mg/ml in the formulation. In an alternate embodiment, the polysaccharide is trimethylchitosan having a concentration of between 1-20 mg/ml in the formulation.

### FIELD OF THE INVENTION

The invention relates to cannabinoid delivery methods, systems and formulations, and particularly to formulations including cannabinoids transmucosal delivery.

### BACKGROUND OF THE INVENTION

A cannabinoid is one of a class of diverse chemical compounds that can influence cannabinoid receptors in humans and other mammals. In general there are three sources of cannabinoids. Endocannabinoids are produced naturally in the body. Phytocannabinoids are produced in plants. One notable source of phytocannabinoids is cannabis sativa L. Synthetic cannabinoids are produced in a laboratory environment. Recently synthetic cannabinoids have been produced by yeast engineered with the ability to produce selected cannabinoids. There are many other ways to synthesize cannabinoids.

Cannabis sativa L includes both marijuana and hemp. Marijuana includes bioactive concentrations of the psychoactive cannabinoid trans-Δ⁹-tetrahydrocannabinol (THC). Hemp typically includes low concentrations of THC, e.g. less than 0.3% THC. Hemp can be rich and other cannabinoids. Over one hundred different phytocannabinoids have been isolated from Cannabis sativa L. Cannabis Sativa includes numerous varieties of hemp, including industrial hemp. Cannabis sativa L also includes numerous species of marijuana including sativa, indica and ruderalis. Each species has a multitude of phenotypes.

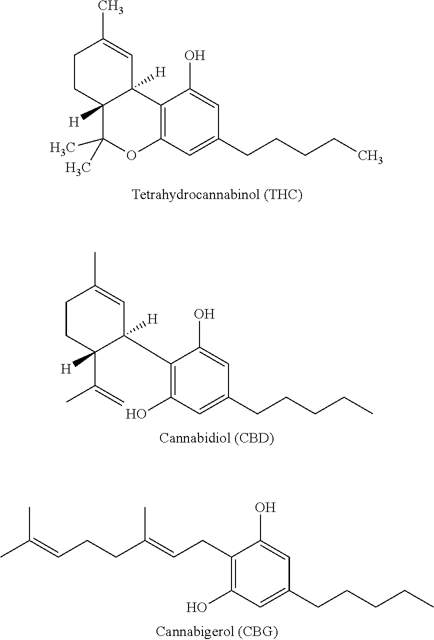

Cannabinoid delivery in humans has traditionally been associated with smoking the flowers of cannabis sativa L. Smoking has several drawbacks however. Smoke may contain small quantities of carcinogenic compounds. Some users simply do not like smoking. Others may have lung conditions which makes smoking very unpleasant. There are benefits to smoking. One is that the cannabinoid are highly bioavailable in a vaporized form.

In the past decade many cannabis products have been developed which are orally ingestible in the form of cannabis essential oil, or an oil-infused edible. While these are highly popular, the bioavailability significantly decreases when cannabinoids are ingested orally. This is primarily due to first pass metabolism. First pass metabolism occurs in the liver. Orally ingested cannabinoids pass-through the digestive tract into the bloodstream and are readily metabolized by the liver. Scientific studies have indicated that up to 97% of THC consumed orally is metabolized through the mechanism of first pass metabolism.

What is desired is a way of delivering cannabinoids to the bloodstream, or to a particular location, in a way that avoids the first pass metabolism and avoids the drawbacks of smoking.

### SUMMARY OF THE INVENTION

The present invention includes a cannabinoid formulation optimized for transmucosal delivery to a human or mammalian subject. The formulation, in one embodiment, includes at least one cannabinoid derived from cannabis sativa L such as CBD or THC. It can be appreciated that the cannabinoids can also be isolated phytocannabinoids from any cannabinoid producing plant, whole plant extracted phytocannabinoids or synthesized.

The invention includes a formulated product including at least one cannabinoid, a first excipient having a lipid profile of at least 40% oleic acid, at least 20% steraic acid, at least 3% linoleic acid, and at least 2% palmitic acid. Preferably the first excipient is shea butter.

Preferably, the formulated product (formulation) includes an active ingredient of at least one cannabinoid selected from the group consisting of tetrahydrocannabinol, cannabidiol, and combinations thereof.

The formulation further includes a second excipient, namely soy lecithin.

The formulation includes a third excipient comprising a bioadhesive polysaccharide. Preferably, the bioadhesive polysaccharide comprises randomly distributed β-(1→4)-linked D-glucosamine and N-acetyl-D-glucosamine to adhere to a mucosal membrane and to simultaneously improve transmucosal delivery of the cannabinoids.

In one embodiment, the polysaccharide is chitosan manufactured by treating the chitin shells of a crustacean in the presence of an alkaline substance such as sodium hydroxide.

In another embodiment, the third excipient has a bioactive amount of a monosaccharide. The monosaccharide is glucosamine in one form or another. In a preferred embodiment, the chitosan includes at least 25% trimethylchitosan, which is a form of glucosamine. In an alternate embodiment, the chitosan includes between 25%-50% trimethylchitosan. In an alternate embodiment, the chitosan includes approximately 50% trimethylchitosan.

### BRIEF DESCRIPTION OF THE DRAWINGS

FIG. 1 is a flow diagram showing a deacetylation process of the present invention.

### DETAILED DESCRIPTION OF THE INVENTION

Shea butter is solid at room temperature and melts rapidly at body temperature. Shea butter has a unique lipid profile. Typically, cold pressed shea butter contains, on a weight to weight basis, oleic acid (40-60%), stearic acid (20-50%), linoleic acid (3-11%), palmitic acid (2-9%), linolenic acid (<1%) and arachidic acid (<1%). This lipid profile provides an optimal lipid profile to enable shea butter to be an excipient for transmucosal delivery of cannabinoids.

Shea butter and lecithin both provide an appropriate lipid profile to enables formation of liposomes to encapsulate a portion of the cannabinoids. This is accomplished with only a minimum amount of kinetic and thermal energy to produce the formulation. Liposome encapsulation can occur in a deliberate manner, but may also occur naturally in response to mixing cannabinoids with the excipients shea butter and lecithin at optimal temperatures.

Chitosan is produced by deacetylation of chitin, which is the structural element in the exoskeleton of crustaceans and cell walls of fungi. On average, the molecular weight of commercially produced chitosan is between 3800 and 20,000 Daltons.

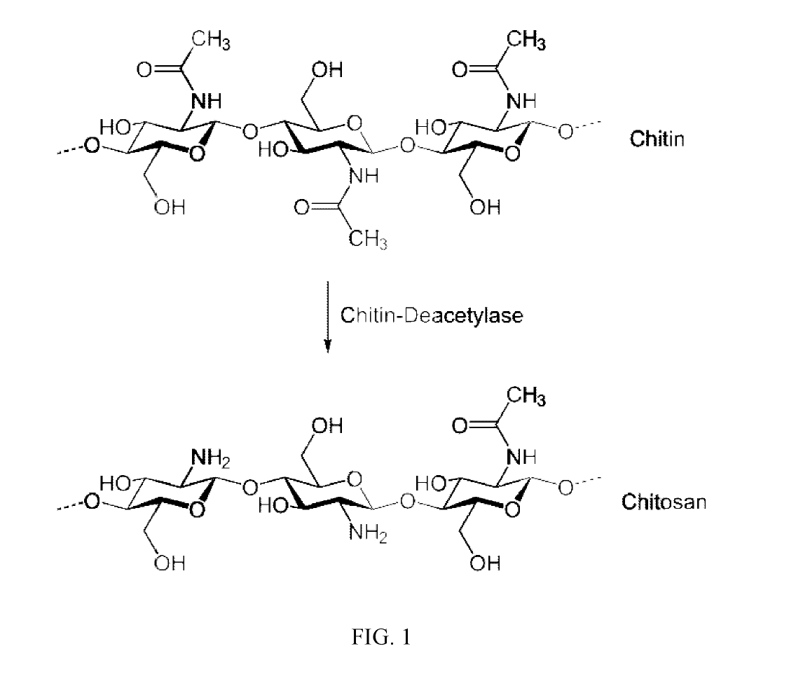

FIG. 1 shows the synthesis of chitosan through molecular diagrams demonstrating the deacetylation process.

Synthesis of chitosan is accomplished by deacetylation of chitin using sodium hydroxide in an aqueous solution. Kinetic control yields a two stage process with the first stage having higher activation energy than the second. For example, the activation energy of the first stage is 48.76 kJ/mol at 25-120° C.

The amino group in chitosan has an acid dissociation constant pKa value of approximately 6.5, which leads to a protonation in both neutral and acidic solutions. The cannabinoids according to the present invention can have acidic or non-acidic forms. In one embodiment, the cannabinoids have a predominant fraction that is in the acidic form, i.e. THC-A.

The charge density of the amino group in chitosan depends on the pH and the % DA-value. Ideally the charge density makes the chitosan water-soluble and an effective bioadhesive which readily binds to negatively charged surfaces such as those found in mucosal membranes. Thus chitosan enhances the latency of delivered cannabinoids in situ on mucosal membranes to optimize the transport of the cannabinoids across epithelial surfaces. Chitosan is a carrier that is both biocompatible and biodegradable.

Chitosan and its derivatives, such as trimethylchitosan can be combined to transfect cancer cells. Transfection of cells typically involves opening transient pores or “holes” in the cell membrane to allow the uptake of material. An increased degree of trimethylation increases the cytotoxicity of cancer cells. In one embodiment, the about 50% of the chitosan is trimethylchitosan. This is an efficient concentration to effectuate liposomal delivery of therapeutic substances such as cannabinoids to cancer cells. In another embodiment, the chitosan includes between 25-50% trimethylchitosan.

Cannabinoids such as THC are known to cause cell aptosis in cancer cells. Oligomeric derivatives of chitosan (3-6 kDa) are relatively nontoxic and have good delivery properties. Accordingly chitosan in combination with appropriate liposomes form an optimal excipient to deliver cannabinoids via a mucosal membrane. In one embodiment, liposomes are derived from soy lecithin, or other lecithin source.

Glucosamine (C₆H₁₃NO₅) is an amino sugar and a prominent precursor in the biochemical synthesis of glycosylated proteins and lipids. Glucosamine is part of the structure of the polysaccharides chitosan and chitin, which compose the exoskeletons of crustaceans and other arthropods, as well as the cell walls of fungi and many higher organisms. Glucosamine is one of the most abundant monosaccharides. It is produced commercially by the hydrolysis of crustacean exoskeletons or, less commonly, by fermentation of a grain such as corn or wheat. In one embodiment, the present invention includes a monosaccharide, such as glucosamine.

Glucosamine is naturally present in the shells of shellfish, animal bones, bone marrow, and fungi. D-Glucosamine is made naturally in the form of glucosamine-6-phosphate, and is the biochemical precursor of many nitrogen-containing sugars. Specifically in humans, glucosamine-6-phosphate is synthesized from fructose 6-phosphate and glutamine by glutamine-fructose-6-phosphate transaminase as the first step of the hexosamine biosynthesis pathway. The end-product of this pathway is uridine diphosphate N-acetylglucosamine (UDP-GlcNAc), which is then used for making glycolipids.

A method of the present invention includes delivering the formulation via the rectal or vaginal cavities of a subject. A variety of health issues can be treated in this way including various forms of cancer or diseases associated with chronic inflammation.

In one method of the invention, manufacturing the formulation including using the shea butter serves as the medium to extract the cannabinoids from the decarboxylated dried cannabis with heat and mechanical agitation in an aqueous solution of shea butter and cannabis biomass. The mixture is cooled to separate the shea butter, which hardens at the surface. The shea butter captures the oil soluble components of the mixture including the cannabinoids yielding whole plant extracted cannabinoids in shea butter.

The method includes formulating the extracted cannabinoids with soy lecithin to increase the bioavailability of the cannabinoids and other compounds during extraction from the cannabis.

The method includes adding premium grade GMP standard chitosan (a natural extract from shellfish shells). This increases mucous membrane permeability while also increasing adhesion of the product to the rectal or vaginal cavity mucous membranes. The adhesion is important to inhibit leakage from either cavity during use, which is inconvenient. The chitosan also improves bioactivity and bioavailability of the cannabinoids.

### Manufacturing Example 1

Heat 4 g dried cannabis to 250 degree F. for 30 minutes to decarboxylate the cannabinoids. Heat 400 g (about 2 cups) of raw unrefined shea butter to melting at approximately 95 degrees F. Add 2 tablespoons of soy lecithin to the shea butter and dissolve. Then add cannabis to shea butter and heat to a temperature of 160 degrees F. for 2 hours. Filter out the cellulosic and other coarse material. Cool the mixture to 100 degrees F. and add 20 mg chitosan per 2 ml and mix thoroughly. Basic formula is complete and can now be either placed into molds by pipette at 2 ml or 5 ml dosing levels or have additional medicinal ingredients added (e.g. essential oils, Ayurvedic compounds, homeopathic remedies, etc.) before being placed into molds and refrigerated yielding a solid molded product.

In one embodiment the mold yields an uncoated product that easily slides into the body due to the high lipid content and the melting point of the shea butter. In another embodiment, the molded product is coated to inhibit melting during handling.

The chitosan can be in a concentration of between 1-20 mg/ml, and preferably between 5-25 mg/ml. In one embodiment, the chitosan is in a concentration of approximately 10 mg/ml.

The cannabinoid mix (the collective amount of all cannabinoids) can be concentrated in a range of 0.01-3% in the formulation. Preferably the cannabinoid mix represents between 0.1%-0.5% of the formulation. In one embodiment, the cannabinoid mix comprises less than 1% of the formulation. These cannabinoid ratios are on a weight to weight basis. Such low concentrations of cannabinoids yield bioactive doses of the formulation because the transmucosal delivery enables most of the cannabinoids to directly enter the blood stream and first pass metabolism degradation of the cannabinoids is avoided. Stated differently a ling dose of cannabinoids in the present formulation can have the effects of a 30-50 mg oral dose.

While the present invention is described by way of example, the true scope of the present invention is expressed in the claims below. Also it can be appreciated that the step of decarboxylation can be adapted to decarboxilate only a portion of the cannabinoids, or none at all to leave the cannabinoids in the acid form. Although the present invention is described in terms of a rectal or vaginal delivery, the present invention can be adapted into a nasal or oral spray, a cosmeceutical, eye drops, or other topical delivery method. Further the present invention can be adapted for oral delivery by increasing the cannabinoid concentrations to 10-60%.

I claim:

1. A cannabinoid formulation for transmucosal delivery comprising:
shea butter;
at least one cannabinoid selected from the group consisting of tetrahydrocannabinol, cannabidiol, and combinations thereof; and
a bioadhesive polysaccharide composed of β-(1→4)-linked D-glucosamine and N-acetyl-D-glucosamine to improve mucosal and cellular permeability.

2. The cannabinoid formulation as set forth in claim 1, wherein the polysaccharide is chitosan in a concentration of between 1-20 mg/ml.

3. The cannabinoid formulation as set forth in claim 2, wherein the chitosan is manufactured by treating the chitin shells of a crustacean with an alkaline substance.

4. The cannabinoid formulation as set forth in claim 3, wherein the alkaline substance is sodium hydroxide.

5. The cannabinoid formulation as set forth in claim 1, further comprising a monosaccharide.

6. The cannabinoid formulation as set forth in claim 1, further comprising glucosamine.

7. The cannabinoid formulation as set forth in claim 1, wherein the chitosan includes at least 25% trimethylchitosan.

8. The cannabinoid formulation as set forth in claim 1, wherein the chitosan includes between 25%-50% trimethylchitosan.

9. The cannabinoid formulation as set forth in claim 1, wherein the chitosan includes approximately 50% trimethylchitosan.

10. A cannabinoid formulation for transmucosal delivery comprising:
shea butter containing at least 40% oleic acid, at least 20% steraic acid, at least 3% linoleic acid, and at least 2% palmitic acid;
at least one cannabinoid selected from the group consisting of tetrahydrocannabinol, cannabidiol, and combinations thereof;
soy lecithin; and
a bioadhesive polysaccharide composed of randomly distributed β-(1→4)-linked D-glucosamine and N-acetyl-D-glucosamine to improve mucosal and cellular permeability.

11. The cannabinoid formulation as set forth in claim 10, wherein the polysaccharide is in chitosan in a concentration of between 5-15 mg/ml in the formulation.

12. The cannabinoid formulation as set forth in claim 11, wherein the chitosan is manufactured by treating the chitin shells of a crustacean with an alkaline substance.

13. The cannabinoid formulation as set forth in claim 12, wherein the alkaline substance is sodium hydroxide.

14. The cannabinoid formulation as set forth in claim 10, further comprising a monosaccharide.

15. The cannabinoid formulation as set forth in claim 10, further comprising glucosamine.

16. The cannabinoid formulation as set forth in claim 10, wherein the chitosan includes at least 25% trimethylchitosan.

17. The cannabinoid formulation as set forth in claim 10, wherein the chitosan includes between 25%-50% trimethylchitosan.

18. The cannabinoid formulation as set forth in claim 10, wherein the chitosan includes approximately 50% trimethylchitosan.

19. A cannabinoid formulation for transmucosal delivery comprising:
shea butter;
at least one cannabinoid selected from the group consisting of tetrahydrocannabinol, cannabidiol, and combinations thereof; and
a bioadhesive polysaccharide to improve mucosal and cellular permeability.

20. The cannabinoid formulation as set forth in claim 19, wherein the polysaccharide is trimethylchitosan in a concentration of between 1-20 mg/ml.
